(19) United States
(12) Patent Application Publication
(10) Pub. No.: US 2026/0133693 A1
(43) Pub. Date: May 14, 2026

(54) SYSTEM AND METHOD FOR STORING AND SHARING GENOMIC DATA USING BLOCKCHAIN

(71) Applicant: CARDIAI TECHNOLOGIES LTD., Calgary (CA)

(72) Inventors: Anmol KAPOOR, Calgary (CA); Sidharth Singh BHINDER, Calgary (CA)

(21) Appl. No.: 19/118,081

(22) PCT Filed: Oct. 3, 2023
(86) PCT No.: PCT/CA2023/051311

§ 371 Date: Apr. 3, 2025

Related U.S. Application Data
(60) Provisional application No. 63/412,585, filed on Oct. 3, 2022.

(51) Int. Cl. G06F 3/06

(52) U.S. Cl. CPC G06F 3/0608; G06F 3/0655; G06F 3/0679

(57) ABSTRACT

A method of compressing genomic data. The method has the steps of: aligning the genomic data with reference data, obtaining difference between the genomic data and the reference data, and compressing the difference using a statistical compression method to obtain compressed genomic data. In some embodiments, the statistical compression method may be an arithmetic coding method. In some embodiments, the method may further has a step of processing the difference using one or more statistical modeling methods, and compressing the processed difference using the statistical compression method. In some embodiments, the method further has a step of assembling a plurality of reads to form the reference data. In some embodiments, the method further has a step of storing compressed genomic data in a blockchain.

### CROSS-REFERENCE TO RELATED APPLICATIONS

This application claims the benefit of U.S. Provisional Patent Application Ser. No. 63/412,585, filed Oct. 3, 2022, the content of which is incorporated herein by reference in its entirety.

### FIELD OF THE DISCLOSURE

The present disclosure relates generally to system and method for storing and/or sharing genomic data such as human genomic data, and in particular to system and method for storing and/or sharing genomic data as compressed archive and/or using blockchain.

### BACKGROUND

Human genomic data carries unique information about an individual and offers unprecedented opportunities for healthcare. The clinical interpretations derived from large genomic datasets may greatly improve healthcare and pave the way for personalized medicine. Genomic data generally requires computer systems and storages for processing and storing due to its huge size (for example, a single human genome takes up 100 gigabytes of storage space). With the rapid advances in genomic sequencing, the amount of genomic data being produced is growing exponentially. Several large-scale sequencing projects for humans and other species are expected to further increase the volume of this data.

However, sharing genomic datasets through computer systems may pose major challenges because, different from traditional medical data, genomic data may indirectly reveal information about descendants and relatives of the data owner and may carry valid information even after the owner passes away. Therefore, stringent data ownership and control measures in computerized human genomic data storage and sharing are required.

Fostering open and responsible genomic-data sharing has constituted a core principle of many national and international initiatives such as the All of Us Research Program in the United States and Global Alliance for Genomics and Health. Despite the widespread attention paid to the importance of genomic-data sharing, technical and governance bottlenecks hinder data sharing. A recent survey of genomic sequencing initiatives demonstrates that lack of conformity and interoperability of bioinformatics pipelines, lack of financial support, together with legal, consent, and privacy related issues are among the major challenges in front of genomic-data sharing.

### SUMMARY

According to one aspect of this disclosure, there is provided a method of compressing genomic data, the method comprising: aligning the genomic data with reference data; obtaining difference between the genomic data and the reference data; and compressing the difference using a statistical compression method to obtain compressed genomic data.

In some embodiments, the statistical compression method is an arithmetic coding method.

In some embodiments, the method further comprises: processing the difference using one or more statistical modeling methods; and said compressing the difference using the statistical compression method comprises: compressing the processed difference using the statistical compression method.

In some embodiments, the method further comprises: assembling a plurality of reads to form the reference data.

In some embodiments, the method further comprises: storing compressed genomic data in a blockchain.

According to one aspect of this disclosure, there is provided one or more non-transitory computer-readable storage devices comprising computer-executable instructions for compressing genomic data, wherein the instructions, when executed, cause a processing structure to perform the above-described method.

According to one aspect of this disclosure, there is provided one or more processor configured for performing the above-described method.

### BRIEF DESCRIPTION OF THE DRAWINGS

For a more complete understanding of the disclosure, reference is made to the following description and accompanying drawings, in which:

FIG. 1 is a schematic diagram of a computer network system for data sharing, according to some embodiments of the present disclosure;

FIG. 2 is a schematic diagram showing a simplified hardware structure of a computing device of the computer network system shown in FIG. 1;

FIG. 3 a schematic diagram showing a simplified software architecture of a computing device of the computer network system shown in FIG. 1;

FIG. 4 is a flowchart showing a process executed by the computer network system shown in FIG. 1 for using a blockchain to store and manipulate (such as insertion and query) genomic data, according to some embodiments of this disclosure;

FIG. 5 shows examples of an example of read, quality scores, and read ID of genomic data;

FIG. 6 is a flowchart showing a reference-based lossless genomic-data compression process executed by the computer network system shown in FIG. 1 for compressing genomic or sequence data while retaining all information from the original file, according to some embodiments;

FIG. 7 is a flowchart showing a reference-based lossless genomic-data compression process executed by the computer network system shown in FIG. 1 for compressing sequence data, according to some other embodiments of this disclosure;

FIG. 8 is a flowchart showing a delta encoding process used at the statistical modeling step of the reference-based lossless genomic-data compression process shown in FIG. 7, according to some other embodiments of this disclosure; and

FIG. 9 is a flowchart showing an assembly-based lossless genomic-data compression process executed by an assembler of the computer network system shown in FIG. 1 for compressing genomic or sequence data while retaining all information from the original file, according to some embodiments.

### DETAILED DESCRIPTION

Some of the challenges associated with genomic-data sharing are rooted in adopting centralized approaches towards genomic-data storage, sharing, and access. The success of centralized data sharing hinges mainly upon functioning and well-resourced central data-storage infrastructures and/or centralized data-access control services. Recent studies reveal that data custodians are confronting constraints in establishing central data-access management mechanisms. Moreover, the non-automated nature of traditional data sharing and access adds to the complexity of oversight on compliance of data sharing and use with the consent forms and data access agreements.

In addition, the centralized platforms are not suitable in facilitating active participation of multiple stakeholders such as individuals and patients in the governance of data sharing.

Distributed networks may be a solution, which enable approved queries to be made to distributed, encrypted databases and allow each independent data contributor to manage the data access. Thus, data sharing based on distributed networks may be more advantageous than that based on third-party's centralized services as distributed networks aim to overcome the inefficiency, expense, and security risks of transferring datasets to central repositories, often across international boundaries.

One of the emerging examples of distributed networks is the Blockchain-based platform for data sharing and access. Blockchain is a decentralized peer-to-peer architecture with nodes of network participants. Each member in the network stores an identical copy of the Blockchain and contributes to the collective process of validating and certifying digital transactions for the network.

According to one aspect of this disclosure, a blockchain system is provided for storing and sharing genomic data (also denoted “sequencing data” or “sequence data” hereinafter) among users. The system disclosed herein offers an alternative to traditional distributed systems with improved data security.

### System Structure

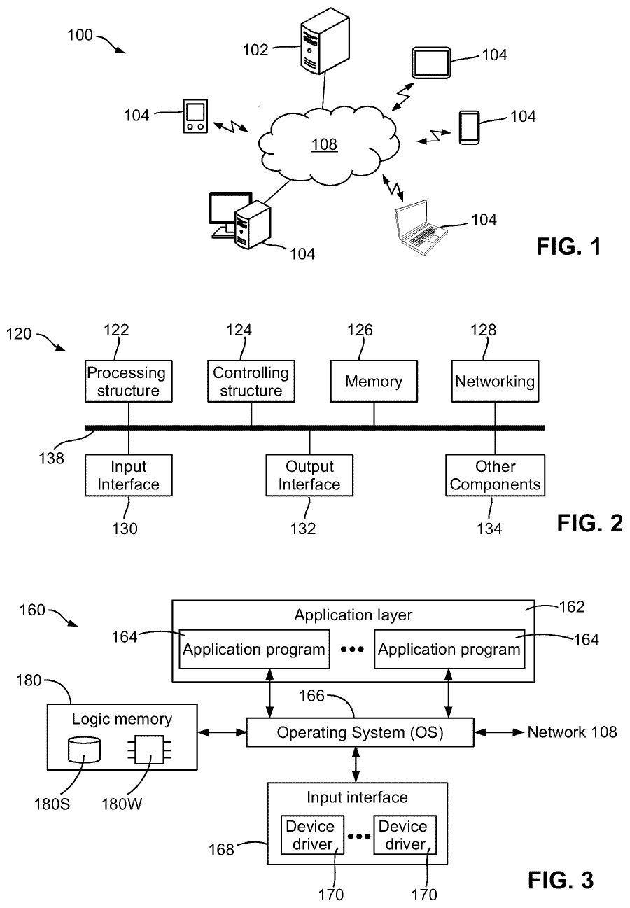

Turning now to FIG. 1, a computer network system for genomic-data storing and sharing is shown and is generally identified using reference numeral 100. As shown, the computer network system 100 comprises one or more server computers 102 and a plurality of client computing devices 104 functionally interconnected by a network 108, such as the Internet, a local area network (LAN), a wide area network (WAN), a metropolitan area network (MAN), and/or the like, via suitable wired and/or wireless networking connections.

The server computers 102 may be computing devices designed specifically for use as a server, and/or general-purpose computing devices acting as server computers while also being used by various users. Each server computer 102 may execute one or more server programs.

The client computing devices 104 may be portable and/or non-portable computing devices such as laptop computers, tablets, smartphones, Personal Digital Assistants (PDAs), desktop computers, and/or the like. Each client computing device 104 may execute one or more client application programs which sometimes may be called “apps”.

Generally, the computing devices 102 and 104 have a similar hardware structure such as a hardware structure 120 shown in FIG. 2. As shown, the computing device 102/104 comprises a processing structure 122, a controlling structure 124, one or more non-transitory computer-readable memory or storage devices 126, a network interface 128, an input interface 130, and an output interface 132, functionally interconnected by a system bus 138. The computing device 102/104 may also comprise other components 134 coupled to the system bus 138.

The processing structure 122 may be one or more single-core or multiple-core computing processors such as INTEL® microprocessors (INTEL is a registered trademark of Intel Corp., Santa Clara, CA, USA), AMD® microprocessors (AMD is a registered trademark of Advanced Micro Devices Inc., Sunnyvale, CA, USA), ARM® microprocessors (ARM is a registered trademark of Arm Ltd., Cambridge, UK) manufactured by a variety of manufactures such as Qualcomm of San Diego, California, USA, under the ARM® architecture, or the like. When the processing structure 122 comprises a plurality of processors, the processors thereof may collaborate via a specialized circuit such as a specialized bus or via the system bus 138.

The processing structure 122 may also comprise one or more real-time processors, programmable logic controllers (PLCs), microcontroller units (MCUs), μ-controllers (UCs), specialized/customized processors and/or controllers using, for example, field-programmable gate array (FPGA) or application-specific integrated circuit (ASIC) technologies, and/or the like.

Generally, each processor of the processing structure 122 comprises necessary circuitries implemented using technologies such as electrical and/or optical hardware components for executing one or more processes to perform various tasks. In many embodiments, the one or more processes may be implemented as firmware and/or software stored in the memory 126. Those skilled in the art will appreciate that, in these embodiments, the one or more processors of the processing structure 122, are usually of no use without meaningful firmware and/or software.

The controlling structure 124 comprises one or more controlling circuits, such as graphic controllers, input/output chipsets, and the like, for coordinating operations of various hardware components and modules of the computing device 102/104.

The memory 126 comprises one or more one or more non-transitory computer-readable storage devices or media accessible by the processing structure 122 and the controlling structure 124 for reading and/or storing instructions for the processing structure 122 to execute, and for reading and/or storing data, including input data and data generated by the processing structure 122 and the controlling structure 124. The memory 126 may be volatile and/or non-volatile, non-removable or removable memory such as RAM, ROM, EEPROM, solid-state memory, hard disks, CD, DVD, flash memory, or the like. In use, the memory 126 is generally divided into a plurality of portions for different use purposes. For example, a portion of the memory 126 (denoted as storage memory herein) may be used for long-term data storing, for example, for storing files or databases. Another portion of the memory 126 may be used as the system memory for storing data during processing (denoted as working memory herein).

The network interface 128 comprises one or more network modules for connecting to other computing devices or networks through the network 108 by using suitable wired and/or wireless communication technologies such as Ethernet, WI-FI® (WI-FI is a registered trademark of Wi-Fi Alliance, Austin, TX, USA), BLUETOOTH® (BLUETOOTH is a registered trademark of Bluetooth Sig Inc., Kirkland, WA, USA), Bluetooth Low Energy (BLE), Z-Wave, Long Range (LoRa), ZIGBEE® (ZIGBEE is a registered trademark of ZigBee Alliance Corp., San Ramon, CA, USA), wireless broadband communication technologies such as Global System for Mobile Communications (GSM), Code Division Multiple Access (CDMA), Universal Mobile Telecommunications System (UMTS), Worldwide Interoperability for Microwave Access (WiMAX), CDMA2000, Long Term Evolution (LTE), 3GPP, 5G New Radio (5G NR) and/or other 5G networks, and/or the like. In some embodiments, parallel ports, serial ports, USB connections, optical connections, or the like may also be used for connecting other computing devices or networks although they are usually considered as input/output interfaces for connecting input/output devices.

The input interface 130 comprises one or more input modules for one or more users to input data via, for example, touch-sensitive screens, touch-sensitive whiteboards, touch-pads, keyboards, computer nice, trackballs, microphones, scanners, cameras, and/or the like. The input interface 130 may be a physically integrated part of the computing device 102/104 (for example, the touch-pad of a laptop computer or the touch-sensitive screen of a tablet), or may be a device physically separated from but functionally coupled to, other components of the computing device 102/104 (for example, a computer mouse). The input interface 130, in some implementation, may be integrated with a display output to form a touch-sensitive screen or a touch-sensitive whiteboard.

The output interface 132 comprises one or more output modules for output data to a user. Examples of the output modules include displays (such as monitors, LCD displays, LED displays, projectors, and the like), speakers, printers, virtual reality (VR) headsets, augmented reality (AR) goggles, and/or the like. The output interface 132 may be a physically integrated part of the computing device 102/104 (for example, the display of a laptop computer or a tablet), or may be a device physically separate from but functionally coupled to other components of the computing device 102/104 (for example, the monitor of a desktop computer).

The computing device 102/104 may also comprise other components 134 such as one or more positioning modules, temperature sensors, barometers, inertial measurement units (IMUs), and/or the like. Examples of the positioning modules may be one or more global navigation satellite system (GNSS) components (for example, one or more components for operation with the Global Positioning System (GPS) of USA, Global'naya Navigatsionnaya Sputnikovaya Sistema (GLONASS) of Russia, the Galileo positioning system of the European Union, and/or the Beidou system of China).

The system bus 138 interconnects various components 122 to 134 enabling them to transmit and receive data and control signals to and from each other.

FIG. 3 shows a simplified software architecture 160 of the computing device 102 or 104. The software architecture 160 comprises an application layer 162, an operating system 166, a logical input/output (I/O) interface 168, and a logical memory 172. The application layer 162, operating system 166, and logical I/O interface 168 are generally implemented as computer-executable instructions or code in the form of software programs or firmware programs stored in the logical memory 172 which may be executed by the processing structure 122.

Herein, a software or firmware program is a set of computer-executable instructions or code stored in one or more non-transitory computer-readable storage devices or media such as the memory 126, and may be read and executed by the processing structure 122 and/or other suitable components of the computing device 102/104 for performing one or more processes. Those skilled in the art will appreciate that a program may be implemented as either software or firmware, depending on the design purposes and requirements. Therefore, for ease of description, the terms “software” and “firmware” may be interchangeably used hereinafter.

Referring back to FIG. 3, the application layer 162 comprises one or more application programs 164 executed by or performed by the processing structure 122 for performing various tasks.

The operating system 166 manages various hardware components of the computing device 102 or 104 via the logical I/O interface 168, manages the logical memory 172, and manages and supports the application programs 164. The operating system 166 is also in communication with other computing devices (not shown) via the network 108 to allow the application programs 164 to communicate with programs running on other computing devices. As those skilled in the art will appreciate, the operating system 166 may be any suitable operating system such as MICROSOFT® WINDOWS® (MICROSOFT and WINDOWS are registered trademarks of the Microsoft Corp., Redmond, WA, USA), APPLE® OS X, APPLE® iOS (APPLE is a registered trademark of Apple Inc., Cupertino, CA, USA), Linux, ANDROID® (ANDROID is a registered trademark of Google Inc., Mountain View, CA, USA), or the like. The computing devices 102 and 104 of the computer network system 100 may all have the same operating system, or may have different operating systems.

The logical I/O interface 168 comprises one or more device drivers 170 for communicating with respective input and output interfaces 130 and 132 for receiving data therefrom and sending data thereto. Received data may be sent to the application layer 162 for being processed by one or more application programs 164. Data generated by the application programs 164 may be sent to the logical I/O interface 168 for outputting to various output devices (via the output interface 132).

The logical memory 172 is a logical mapping of the physical memory 126 for facilitating the application programs 164 to access. In this embodiment, the logical memory 172 comprises a storage memory area that may be mapped to a non-volatile physical memory such as hard disks, solid-state disks, flash drives, and/or the like, generally for long-term data storage therein. The logical memory 172 also comprises a working memory area that is generally mapped to high-speed, and in some implementations, volatile physical memory such as RAM, generally for application programs 164 to temporarily store data during program execution. For example, an application program 164 may load data from the storage memory area into the working memory area, and may store data generated during its execution into the working memory area. The application program 164 may also store some data into the storage memory area as required or in response to a user's command.

In a server computer 102, the application layer 162 generally comprises one or more server-side application programs 164 which provide(s) server functions for managing network communication with client computing devices 104 and facilitating collaboration between the server computer 102 and the client computing devices 104. Herein, the term “server” may refer to a server computer 102 from a hardware point of view, or to a logical server from a software point of view, depending on the context.

As described above, the processing structure 122 is usually of no use without meaningful firmware and/or software. Similarly, while a computer system 100 may have the potential to perform various tasks, it cannot perform any tasks and is of no use without meaningful firmware and/or software. As will be described in more detail later, the computer system 100 described herein, as a combination of hardware and software, generally produces tangible results tied to the physical world, wherein the tangible results such as those described herein may lead to improvements to the computer and system themselves.

### Blockchain System

In some embodiments, the computer network system 100 is a blockchain system which may be a public blockchain system accessible to the public, or a private blockchain system across one or more organizations or one or more industries.

The blockchain system 100 may be considered a decentralized and distributed database system wherein the database is managed by a plurality of users via any suitable computing devices (such as a plurality of server computers 102 and/or a plurality of client computing devices 104). More specifically, the computer network system 100 maintains a decentralized, distributed digital ledger of transactions (a so-called “blockchain network” or simply a “blockchain”). Thus, there is no central point of control of the computer network system 100 and the blockchain is stored in a plurality of physical computing devices 102/104 in the computer network system 100. The server computers 102 in the computer network system 100 are generally in similar roles as the client computing devices 104 in maintaining the blockchain except that the server computers 102 may also be responsible for computer-network managements of the computer network system 100.

The blockchain is duplicated and distributed across the entire computer network system 100 and is shared in a peer-to-peer fashion. The blockchain comprises a plurality of blocks linked by cryptographic hashes. Each block comprises an index, a timestamp, a cryptographic hash of the content of the previous block, a hash of its content, and transaction data which is about the state of the network and stored in the root of a trie (which is a type of k-ary search tree; also called a digital tree or a prefix tree). Moreover, various blockchain technologies use various cryptographic proof methods, such as proof of work, proof of stake, and the like, as part of their consensus mechanisms to achieve agreement on a data value or a state of the network among distributed processes in the computer network system 100.

In these embodiments, the computer network system 100 uses the Solana (SOL) blockchain technology developed by Solana Labs & Solana Foundation. The state trie data structure in Solana blockchain stores information of users and SOL contract accounts. The Solana blockchain uses proof of stake consensus enhanced by the proof of history method.

The Solana blockchain comprises conditional programs (the so-called “smart contracts”) for automation such as automating the execution of an agreement, automating a workflow, automatically triggering the next action when conditions are met, and/or the like. A smart contract comprises one or more predefined conditions and is executed when the predefined conditions thereof are met. In these embodiments, the code of a smart contract is stored at an address in network storage, and also maintains its own storage (such as for storing variables).

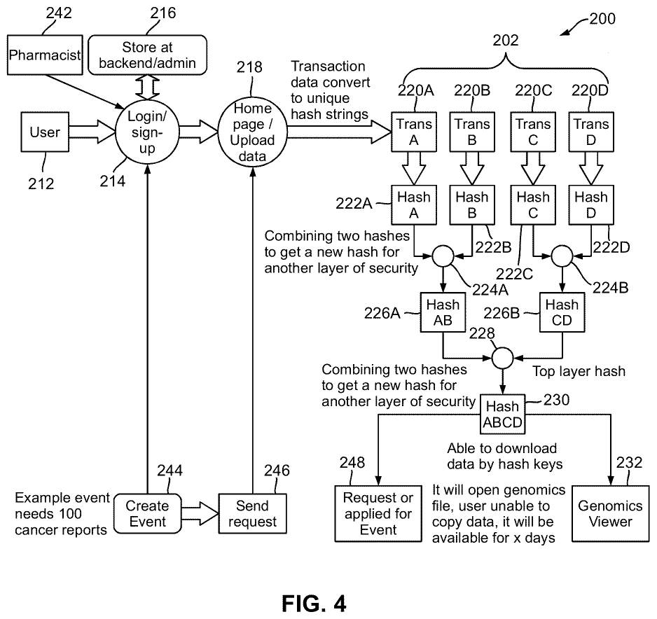

FIG. 4 is a flowchart showing a process 200 executed by the computer network system 100 (or more specifically, one or more processors 122 thereof) for using a blockchain to store and manipulate (such as insertion and query) genomic data, according to some embodiments of this disclosure. For illustrative purposes, the example shown in FIG. 4 comprises four nodes 202 (such as four client computing devices 104) each syncing a copy of the blockchain.

As shown in FIG. 4, a user 212 may login to the computer network system 100 (step 214) wherein the user credentials (such as username and password) may be stored at the backend 216 (such as a server 102) and may be managed by authorized users such as system administrators. A new user 212 may also sign up by setting the user's credentials at the backend 216.

After logging in, the user 212 may use a homepage to upload the user's genomic data (step 218). The uploaded genomic data is processed as transaction data which may be, for example, converted to unique hash strings in the four nodes 202. More specifically, each node 202 receives a copy of the transaction data (220A to 220D) and converts its copy of the transaction data to a unique hash string 222A to 222D. Then, the hash strings 222A to 222D are partitioned into one or more hash-string pairs (for example, hash-string pair 222A/222B, and has-string pair 222C/222D) and each pair of hash strings are combined (step 224) to obtain a new hash string (226A, 226B) for another layer of security.

The pairing and combing of hash strings may be repeated. For example, as shown in FIG. 4, the two hash strings 226A and 226B are further paired and combined (step 228) to obtain a top-layer hash string 230 which may be used for retrieving the genomic data stored in the blockchain. For example, a genomic data of the user 212 stored in the computer network system 100 may be sent to a genomics viewer 232 under the viewing request of another user 242 such as a pharmacist. As another example, in case an event 244 is created (such as requiring 100 cancer reports), a request 244 is sent (step 246) to the homepage 218. Then, the top-layer hash string 230 may be used to retrieve (step 248) the requested genomic data from the blockchain for responding to the event 244.

In some embodiments, the retrieved genomic data is only for viewing in the genomics viewer 232 or for responding to the event 244 for a predefined time period (for example, within a predefined number of days), and/or without being copied out of the blockchain without the permission of the owner of the genomic data.

In the following, the details of various technical features of the computer network system 100 are described.

### Genomic Data Storage

In some embodiments, the genomic data is stored in the blockchain as binary alignment and map (BAM) files. A BAM file is a binary file that stores sequence alignment data, and may be used for application-specific analyses. The BAM files are saved in different transactions which are associated with a common hash string as shown in FIG. 4.

### Calculation of Genomic Data

In some embodiments, the blockchain ledger uses the following for storage calculations:
All calculations are in actual bytes, that is, 1 kilobytes (KB)=1024 bytes.
All ledger blocks are 1 megabyte (MB).
Only hash, signature, or key data is stored in the blockchain ledger.

In some embodiments, the computer network system 100 has about 1000 transactions per block. With above parameters, the amount of storage required per Transactions-Per-Second (TPS) is about 6.75 gigabytes (GB) or 0.00659 tebibyte (TiB)/transaction/year.

### Genomics Viewer

In some embodiments, the genomics viewer 232 provides following feature:
Opening genomic data in human-readable manner;
Opening and displaying requested genomic data under the condition of meeting all permissions and date requirements;
Downloading the displayed genomic data to a local computing device under the mission of the owner of the genomic data;
Uploading genomic data; and
A browser based viewer.

### Non-Fungible Token (NFT)

Non-fungible tokens (NFTs) are cryptographically unique tokens that are linked to digital (and sometimes physical) content, providing proof of ownership. NFTs have been used in many areas such as artwork, digital collectibles, music, items in video games, and the like.

In some embodiments, the computer network system 100 may store the genomics data as NFTs. For example, the computer network system 100 may use an avatar with a background as a unique transaction identifier (ID) for a user to upload genomics data and create a new NFT token (associated with the unique transaction ID). A photo of the user may be used for uniquely identifying the user at the frontend.

### Participatory Access Control

One of the main challenges in managing genomic-data sharing is access control. As genomic data may reveal sensitive health- and non-health-related personal information, adopting privacy-preserving mechanisms when sharing data is imperative. Conventionally, the access control has been managed in non-automated ways, mainly through access committees who vet the eligibility of data users and allow access to specific datasets or access to the data available in the database. However, the major shortcomings are associated with such controlled access models, including lack of harmonization in access policies, burdensome or bureaucratic access procedures, resource-intensive monitoring, lack of adequate tools for ongoing oversight, and/or the like.

In some embodiments, the computer network system 100 uses a “permissioned” structure of Blockchain for managing and controlling the access to sensitive genomic data in an automated way, wherein only pre-approved users are allowed access to the genomic data. More specifically, the computer network system 100 provides metadata of the genomic datasets (which is a description of the genomic data rather than the genomic data itself) and allows all users of the computer network system 100 to access the metadata. On the other hand, the computer network system 100 uses the pseudo anonymity and public key infrastructure (PKI) to encrypt the content of the blockchain (which comprises the genomic data) and only allow authorized users to access.

Thus, the computer network system 100 allows discoverability of the existing genomic datasets while protecting the privacy of the individuals by restricting access of the actual genomic data to authorized users. In addition, in order to ensure anonymity of the genomic datasets when necessary, the computer network system 100 may use suitable tools such as software guard extension (SGX) for leveraging multiple cryptographic protocols to enable efficient and secure data storage and computation outsourcing.

In some embodiments, the computer network system 100 may further use other suitable methods such as maintaining sensitive data off-chain to further protect privacy of sensitive data.

In some embodiments, the computer network system 100 may manage the access control in a participatory way, wherein individuals (that is, the genomic-data subjects), healthcare professionals, and researchers may participate in managing the gnomic-data access control. In these embodiments, the computer network system 100 provides the necessary infrastructure in which various stakeholders may be directly involved in the management of gnomic-data access. Thus, the computer network system 100 creates the opportunity for medical information to remain the property of the patient (that is, the genomic-data subject), and allows an individual to opt in or out of specific events (such as research studies).

In some embodiments, the computer network system 100 may use biometric data of a patient as a private key, requiring other users to gain approval and ascent from the patient before using their anonymized health data.

### Data Ownership and DNA Coins

As those skilled in the art understand, there is an increasing support from scholars, patient groups, and the general public to recognize the individual's ownership rights on raw genomic data and facilitate the access to raw genomic data and related medical records.

In some embodiments, the computer network system 100 allows individuals to maintain ownership of their personal genomic- and health-related data, and decide how to share their data and under what conditions. As a result, individuals may share their genomic data for various purposes.

Thus, the computer network system 100 ensures that users may share their genomic data with privacy-preserving methods that facilitate compliance to legal and ethical standards, which may be beneficial in offering various approaches to address governance challenges in genomic-data sharing.

Consequently, the computer network system 100 enables governing open networks, in which the potentials of decentralized networks, industry needs, and consumer genetics are being harvested. The computer network system 100 aims to scale the amount of data, while providing various models of ownership and facilitating active participation of individuals in the governance of data sharing.

In particular, the computer network system 100 may automate the procedure of data access control and improve the transparency and fairness in genomic data access. Similarly, enforceability of access agreements may be significantly improved by using smart contracts of the computer network system 100, which may provide reassurance for different users such as researchers and data custodians that downstream data uses may be in compliance with the terms and conditions of data uses.

Moreover, with the computer network system 100, the role of patients and individuals in the data-sharing ecosystem may be strengthened, and the monopoly of the public and private test providers on management of the genomic-data sharing may be diminished. Blockchain has the ability to create new commons that occupy a space between the market and public goods.

### Reference-Based Compression of Genomic Data

As those skilled in the art will appreciate, the amount of genomic data is usually huge. With the development of next-generation sequencing (NGS) technology, researchers have had to adapt quickly to cope with the vast increase in raw genomic data. Experiments previously conducted with microarrays and resulted in several megabytes of data are now performed by sequencing, producing many gigabytes of data, and demanding a significant investment in computational infrastructure. While the cost of disk storage has steadily decreased over time, it has not matched the dramatic change in the cost and volume of sequencing. A transformative breakthrough in storage technology may occur in the coming years, but the era of the $1,000 genome is certain to arrive before that of the $100 petabyte hard disk.

As cloud computing and software-as-a-service (Saas) become increasingly relevant to molecular biology research, hours spent transferring NGS datasets to and from off-site servers for analysis will delay meaningful results. More often researchers will be forced to maximize bandwidth by physically transporting storage media (so-called the “sneakernet”), which is an expensive and logistically complicated option. These difficulties will only be amplified as exponentially more sequencing data are generated, implying that even moderate gains in domain-specific compression methods will translate into a significant reduction in the cost of managing these massive data sets over time.

Thus, compression techniques play an important role in enabling efficient storage and transfer of genomic data due to its large size. However, traditional general-purpose data-compression methods and programs such as Gzip may not fully exploit the inherent redundancy in genomic data and thus may not achieve high compression rate. Furthermore, in many cases the genomic data may be noisy, and it is possible to use lossy compression methods that can reduce the storage space without significant adverse impacts on the data quality for downstream analysis.

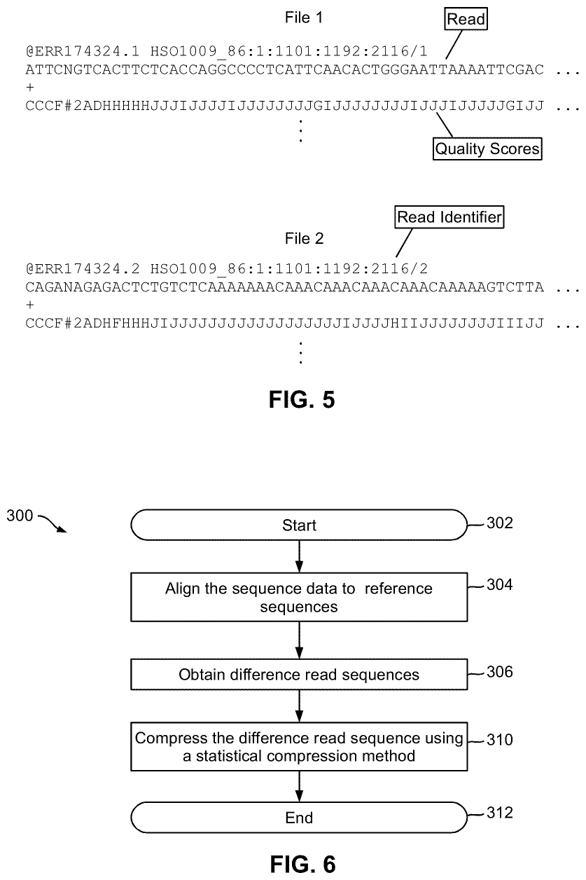

Storage and analysis of NGS data centers primarily around two formats that have arisen recently as de facto standards, that is, the FASTQ format and the sequence alignment map (SAM) format. A FASTQ file stores, in addition to nucleotide sequences, a unique ID for each read (denoted “read ID”; which is the DNA sequence from one fragment, that is, a small section of DNA) and quality scores, which encode estimates of the probability that each base is correctly called. The SAM format is far more complex but also more tightly defined, and comes with a reference implementation in the form of SAMtools. It is able to store alignment information in addition to read IDs, sequences and quality scores. SAM files, which are stored in plain text, can also be converted to the BAM format, a compressed binary version of SAM, which is far more compact and allows for relatively efficient random access. FIG. 5 shows an example of read, quality scores, and read ID.

Compression of nucleotide sequences has been the target of some interest, but compressing NGS data, made up of millions of short fragments of a greater whole, combined with metadata in the form of read IDs and quality scores, presents a very different problem and demands new techniques. Splitting the data into separate contexts for read IDs, sequences, and quality scores, and compressing them with the Lempel-Zip algorithm and Huffman coding has been explored, which demonstrates the promise of domain-specific compression with significant gains over general-purpose programs such as gzip and bzip2.

Reference-based compression methods exploits the redundant nature of the data by aligning reads to a known reference genome sequence and storing genomic positions in place of nucleotide sequences. Decompression is then performed by copying the read sequences from the genome. Though any differences from the reference sequence must also be stored, referenced-based methods can achieve much higher compression with increasing efficient for long read lengths, because storing a genomic position requires the same amount of space, regardless of the length of the read.

For some applications, reference-based compression may be improved by storing only single nucleotide polymorphism (SNP) information, summarizing a sequencing experiment in several megabytes. However, discarding the raw reads would prevent any reanalysis of the data.

While a reference-based method typically results in superior compression, it has a number of disadvantages. Most evidently, an appropriate reference sequence database is not always available, particularly in the case of metagenomic sequencing. One could be contrived by compiling a set of genomes from species expected to be represented in the sample. However, a high degree of expertise is required to curate and manage such a project-dependent database. Secondly, there is the practical concern that files compressed with a reference-based approach are not self-contained. Decompression requires precisely the same reference database used for compression, and if the reference database is lost or forgotten, the compressed data becomes inaccessible.

Another method of short read compression is lossy encoding of sequence quality scores. This follows naturally from the realization that quality scores are particularly difficult to compress. Unlike read IDs which are highly redundant, or nucleotide sequences which contain some structure, quality scores are inconsistently encoded between protocols and computational pipelines and are often simply high-entropy. It is dissatisfying that metadata (such as quality scores) should consume more space than primary data (such as nucleotide sequences). Yet, also dissatisfying to many researchers is the thought of discarding information without a very good understanding of its effect on downstream analysis.

Decreasing the entropy of quality scores while retaining accuracy is an important goal. However, successful lossy compression demands an understanding of what is lost. For example, lossy audio compression (such as MP3) is grounded in psychoacoustic principles, preferentially discarding the least perceptible sound. Conjuring a similarly principled method for NGS quality scores is difficult given that both the algorithms that generate them and the algorithms that are informed by them are moving targets.

In the following a reference-based lossless compression method is described, which leverages a variety of techniques to achieve very high compression over sequencing data of many types, yet remains efficient and practical. Given aligned reads in SAM or BAM format, and the reference sequence to which they are aligned (in FASTA format), the reference-based lossless compression method compresses the reads while preserving all information in the SAM/BAM file (including the header, read IDs, alignment information, and all optional fields allowed by the SAM format. Unaligned reads are retained and compressed using a Markov chain model.

As will be described in more detail later, the reference-based lossless compression method only stores the unaligned reads rather than the whole genome. With the use of statistical-modeling based initial compression and a statistical compression method, the reference-based lossless compression method may effectively compress very large sequence data to less than 15% of their original size with no loss of information.

FIG. 6 is a flowchart showing a reference-based lossless genomic-data compression process 300 executed by the computer network system 100 (or more specifically, one or more processors 122 thereof) for compressing genomic or sequence data while retaining all information from the original file, according to some embodiments. The sequence data may be stored in any suitable format such as in the FASTQ and SAM/BAM formats (for example, as NGS data in the FASTQ and SAM/BAM formats).

After the process starts (step 302), the computer network system 100 aligns the sequence data to a reference sequence data (step 304), and obtains one or more difference read sequences which comprises the sequence data that is different to the reference sequence data (step 306). The difference read sequences are then stored as positions within the assembled contigs. Herein, a contig is a set of DNA segments or sequences that overlap in a way that provides a contiguous representation of a genomic region.

At step 310, a statistical compression method is used for compressing read IDs, quality scores, alignment information, read sequences, and/or the like. The process 300 then ends (step 312).

In some embodiments, the statistical compression method used in the process 300 is an arithmetic coding method, which is a form of entropy coding approaching optimality. Generally, when encoding a string using the arithmetic coding method, characters with more frequent occurrences (or higher occurrence probabilities) are stored with fewer bits and characters with less frequent occurrences (or lower occurrence probabilities) are stored with more bits, thereby resulting in reduced number of bits in total. Unlike Huffman coding (which is another form of entropy coding), arithmetic coding methods have the advantage that they can assign codes of a non-integral number of bits. For example, if a symbol appears with probability 0.1, it can be encoded near to its optimal code length of −log₂(0.1)=3.3 bits.

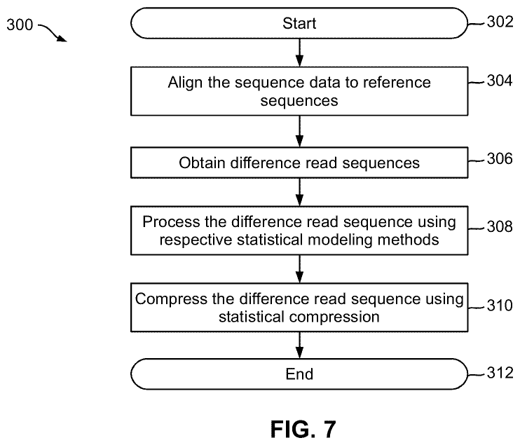

FIG. 7 is a flowchart showing a reference-based lossless genomic-data compression process 300 executed by the computer network system 100 (or more specifically, one or more processors 122 thereof) for compressing sequence data, according to some other embodiments of this disclosure. The process 300 in these embodiments are similar to that shown in FIG. 6. However, by leveraging the advantage of arithmetic coding that it allows a complete separation between statistical modeling and encoding, the process 300 in these embodiments further comprises a statistical modeling step 308 before the encoding step 310. In other words, the computer network system 100 processes the difference read sequences using respective statistical modeling methods for an initial compression of the sequence data (step 308) and then uses the statistical compression method to further compress the sequence data (step 310) thereby achieving increased compression ratio.

In some embodiments, the computer network system 100 uses the same statistical compression method (such as the same arithmetic coder) to encode various fields of the sequence data such as quality scores, read IDs, nucleotide sequences, alignment information, and/or the like, the computer network system 100 may use different statistical models for initial compression of different fields.

Those skilled in the art will appreciate that, in various embodiments, each field of the sequence data may not necessarily need to be initially compressed using a different statistical model. Rather, depending on the implementation, some fields of the sequence data may be initially compressed at step 308 using different statistical models, some other fields of the sequence data may be initially compressed at step 308 using the same statistical model, and/or yet some other fields of the sequence data may not be initially compressed at step 308.

By using statistical modeling at step 308 and statistical compression at step 310, the process 300 achieves a tremendous advantage over general-purpose compression methods that lump everything into a single context. In some embodiments, the statistical models are adaptive models with parameters trained and updated as data are compressed, so that an increasingly tight fit and high compression ratio is achieved on large files.

### Statistical Modeling for Read Identifiers

Read IDs uniquely identify the reads. While integers may be used as read IDs, typically each read is associated with a complex string containing the instrument name, run identifier, flow cell identifier and tile coordinates. Much of this information is the same for every read and is simply repeated, thereby introducing redundancy.

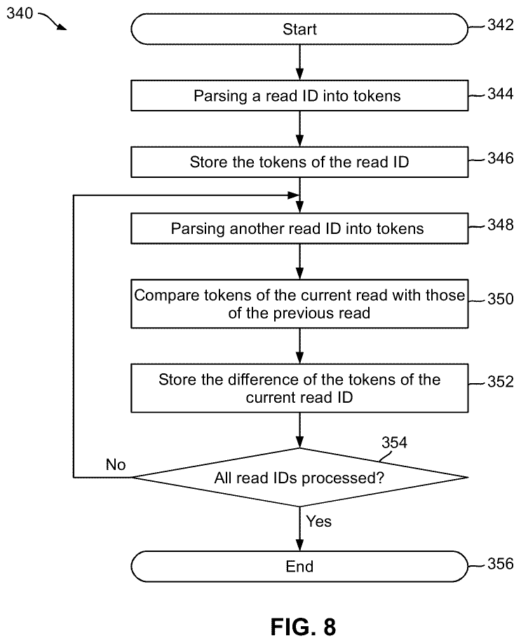

In some embodiments, a delta encoding method may be used to remove this redundancy. FIG. 8 is a flowchart showing the delta encoding process 340.

After the process 340 starts (step 342), a parser (which may be an application program or program module 164) tokenizes a read ID into separate tokens (step 344), and stores the tokens of the read ID (step 346). Then, the parser tokenizes another read ID into tokens (step 348), and compares the tokens of the current read ID with those (that is, tokens in the same positions) of the previous read ID (step 350). At step 352, non-identical tokens (that is, the tokens having values different from those of the previous read ID) are stored wherein identical tokens (that is, the tokens having the same values as those of the previous read ID) are simply marked. The parser then checks if all read IDs are processed (step 354). If all read IDs are processed, the process 340 ends; otherwise, the process 340 goes back to step 348 to tokenize another read ID.

At step 352, numerical non-identical tokens (that is, non-identical tokens having numerical values) may be efficiently stored either directly or as an offset from those of the previous read ID. Non-numerical non-identical tokens (that is, non-identical tokens having non-numerical values) may be encoded by partitioning the non-identical token into an identical portion (that is a portion thereof identical to that of the corresponding token of the previous read ID) such as an identical prefix and a non-identical portion such as a non-identical suffix, and stores the non-identical suffix.

By using the delta encoding method and the arithmetic coding method, tokens that remain the same from read to read (for example, the instrument name) may be compressed to a small or even negligible amount of space (for example, codes of less than one (1) bit for such tokens). As a result, read IDs, which are often 50 bytes or longer, are typically stored in two (2) to four (4) bytes after compression. Notably, in reads produced from Illumina® instruments (Illumina is a registered trademark of Illumina, Inc. of San Diego, CA, USA), most parts of the read IDs can be compressed to consume almost no space; the remaining few bytes are accounted for by tile coordinates which are almost never needed in downstream analysis, and removing them as a preprocessing step may further reduce sizes of compressed sequence data.

### Statistical Modeling for Nucleotide Sequences

In some embodiments, a statistical model based on high-order Markov chains (such as an order-12 Markov chain) is used for compressing nucleotide sequences of the difference read sequences, wherein, the nucleotide at a given position in a read may be predicted using, for example, the preceding 12 positions. While the statistical model used in these embodiments may use more memory than traditional general-purpose compression methods (for example, 4¹³=67,108.864 parameters may be needed, each represented in 32 bits), it is simple and extremely efficient (very little computation is required and run time is limited primarily by memory latency as lookups in such a large table result in frequent cache misses).

While the order-12 Markov chain may be less adaptive to compression of extremely short files, after compressing a plurality of reads (such as several million reads), the parameters of the order-12 Markov chain may be tightly fit to the nucleotide composition of the sequence data so that the remaining reads may be highly compressed. Thus, compressing large files results in a tight fit and high compression.

### Statistical Modeling for Quality Scores

The quality score at a given position is highly correlated with the score at the preceding position. Thus, in some embodiments, a Markov chain is used as a statistic model for initial compression of the quality scores. However, unlike nucleotides, quality scores are over a much larger alphabet (typically 41 to 46 distinct scores), which limits the order of the Markov chain as long chains require a great deal of space and take a unrealistic amount of data to train.

In some embodiments, to reduce the number of parameters, an order-3 Markov chain is used with coarsely binning or storing the distal two positions. In addition to the preceding three positions, the computer network system 100 conditions on the position within the read and a running count of the number of large jumps in quality scores between adjacent positions (where a “large jump” is defined as |qᵢ−qᵢ₋₁|>1), which allows reads with highly variable quality scores to be encoded using separate models. Both of these variables are binned or stored to control the number of parameters.

### Assembly-Based Compression of Genomic Data

In some embodiments, the computer network system 100 may use a de novo assembly method for compressing genomic data, which uses a probabilistic data structure to dramatically reduce the memory required by traditional de Bruijn graph assemblers, allowing millions of reads to be assembled very efficiently. Read sequences are then stored as positions within the assembled contigs. This is combined with statistical compression of read IDs, quality scores, alignment information, sequences, and/or the like, effectively compressing very large data sets to less than 15% of their original size with no loss of information. Compared to the reference-based compression methods, the assembly-based compression method disclosed herein requires no external sequence database and produces entirely self-contained files.

Roughly, the assembly-based compression method disclosed herein may be considered similar to the Lempel-Ziv algorithm, wherein as sequences are read, they are matched to previously observed data, which may be contigs assembled from previously observed data (these contigs are not explicitly stored, but rather reassembled during decompression).

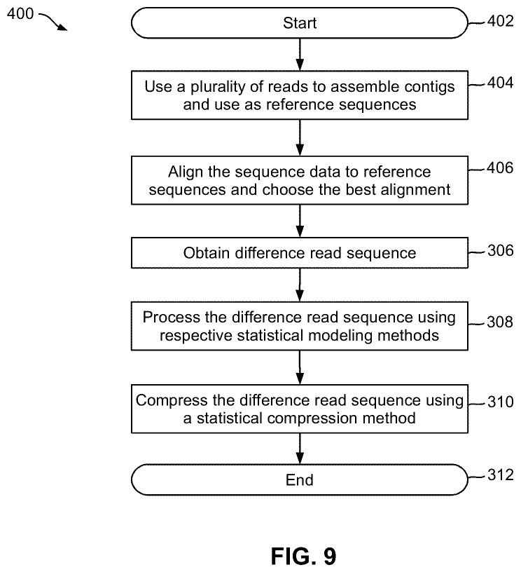

FIG. 9 is a flowchart showing the assembly-based lossless genomic-data compression process 400 executed by an assembler (which may be an application program or program module 164) of the computer network system 100 (or more specifically, one or more processors 122 thereof) for compressing genomic or sequence data while retaining all information from the original file, according to some embodiments. The sequence data may be stored in any suitable format such as in the FASTQ and SAM/BAM formats (for example, as NGS data in the FASTQ and SAM/BAM formats).

After the process starts (step 402), the computer network system 100 use a plurality of reads (such as the first 2.5 million reads) to assemble contigs which are then used as reference sequences to encode aligned reads compactly as positions (step 404).

Once contigs are assembled, read sequences are aligned using a simple “seed and extend” method (step 406). At this step, 12-mer seeds are matched using a hash table, and candidate alignments are evaluated using Hamming distance. The best alignment (for example, the one with the lowest Hamming distance) is chosen, which may fall below a given cutoff. The read is then encoded as a position within the contig set.

This alignment method is simple and fast, and is effective on platforms in which erroneous indels occur infrequently (such as Illumina). After alignment, the rest of the process 400 may be similar to that shown in FIG. 6 (comprising steps 306 and 310) or that shown in FIG. 7 (comprising steps 306, 308, and 310).

Traditionally, de novo assembly is computationally intensive. The most commonly used technique involves constructing a de Bruijn graph, a directed graph in which each vertex represents a nucleotide k-mer present in the data for some fixed k (for example, k=25). A directed edge from a k-mer u to v occurs if and only if the (k−1)-mer suffix of u is also the prefix of v. In principle, given such a graph, an assembly may be produced by finding an Eulerian path, that is, a path that following each edge in the graph exactly once. In practice, since NGS data has a non-negligible error rate, assemblers augment each vertex with the number of observed occurrences of the k-mer and leverage these counts using a variety of heuristics to filter out spurious paths.

A significant bottleneck of the de Bruijn graph approach is building an implicit representation of the graph by counting and storing k-mer occurrences in a hash table. The assembler implemented in Quip overcomes this bottleneck to a large extent by using a data structure based on the Bloom filter to count k-mers. The Bloom filter is a probabilistic data structure that represents a set of elements extremely compactly, at the cost of elements occasionally colliding and incorrectly being reported as present in the set. It is probabilistic in the sense that these collisions occur pseudo-randomly, determined by the size of the table and the hash functions chosen, but generally with low probability.

The Bloom filter is generalized in the counting Bloom filter, in which an arbitrary count may be associated with each element. The d-left counting Bloom filter (dICBF) is a refinement of the counting Bloom filter requiring significantly less space to achieve the same false positive rate. In some embodiments, the assembler of the computer network system 100 is implemented based on a realization of the dICBF. As the assembler uses a probabilistic data structure, k-mers are occasionally reported to have incorrect (inflated) counts. While the assembly can be made less accurate by these incorrect counts, a poor assembly only results in slightly reduced compression ratio. Compression remains lossless regardless of the assembly quality, and in practice collisions in the dICBF occur at a very low rate.

Given a probabilistic de Bruijn graph, the assembler of the computer network system 100 assembles contigs using a simple and efficient greedy method. A read sequence is used as a seed and extended on both ends one nucleotide at a time by repeatedly finding the most abundant k-mer that overlaps the end of the contig by k−1 bases. More sophisticated heuristics may also be used.

### Memory Efficiency

Memory efficient assembly has been a goal of particular interest and is a topic of ongoing research. In prior art, efficient means have been developed for representing de Bruijn graphs using sparse bitmaps compressed with Elias-Fano encoding. The String Graph Assembler relies on the FM-index to build a compact representation of the set of short reads from which contigs are generated by searching for overlaps. Both of these methods sacrifice time-efficiency (significantly longer run times than traditional assemblers) for improving space-efficiency.

The assembly-based compression method disclosed herein does not used exact representations and rather relies on a probabilistic data structure. The assembly-based compression method disclosed herein uses the Bloom filter to store k-mers occurring only once, reducing the memory required by the hash table.

### Metadata

In some embodiments, the sequence-data compression methods disclosed herein used by the computer network system 100 provide several useful features to protect data integrity. First, the output (also denoted the “archive”) of the sequence-data compression method is divided into blocks of several megabytes each. In each block a separate 64-bit checksum is computed for read IDs, nucleotide sequences, and quality scores. When the archive is decompressed, these checksums are recomputed on the decompressed data and compared with the stored checksums, verifying the correctness or integrity of the archive.

Apart from data corruption, reference-based compression methods may have the possibility of data loss if the reference used for compression is lost, or an incorrect reference is used. To protect against the loss of reference, the archive file of the computer network system 100 (that is, the compressed sequence data file) stores a 64-bit hash of the reference sequence, ensuring that the same sequence is used for decompression. To assist in locating the correct reference, the file name, and the lengths and names of the sequences used in compression are also stored without compression so that they are accessible without decompression.

Additionally, block headers store, without compression, the number of reads and bases compressed in the block, allowing summary statistics of a sequence dataset to be listed without decompression.

### Modes of FASTQ Compression

In some embodiments, the computer network system 100 supports the following recommended modes of FASTQ compression:
1. Lossless mode (default): In this mode, the FASTQ file is compressed so that it can be exactly reconstructed, that is, the reads, quality, read identifiers, and the read order information can be perfectly recovered.
2. Recommended lossy mode: In this mode, the information relevant for most of the genomic applications (such as alignment, assembly, variant calling, and the like) is preserved. This includes the reads along with pairing information and binned quality values. The quality values are subjected to the Illumina's standardized 8-level binning (https:/www.illumina.com/documents/products/whitepapers/whitepaper_datacompression.pdf) before compression (Novaseq qualities are left unchanged). The read identifiers and the order of the pairs is discarded (that is, the decompressed FASTQ file contains the read pairs in an arbitrary order). The relative ordering of the first and the second read in each pair is still preserved. The sequence data compression may be highly customized based on the user needs, and provide additional capabilities such as custom binning of quality values using QVZ (which is a lossy compression method) and binary thresholding. For short reads (up to 511 bp), the read compression may be based on hash-based read compressor (HARC) with significant improvements and added support for variable-length reads. The sequence data compression also supports long read compression, where block sorting compressor (BSC; https://github. com/IlyaGrebnov/libbsc/) is used as the read compressor. Furthermore, the sequence-data compression method disclosed herein compresses the streams in blocks, allowing for fast decompression of a subset of reads (random access).

### (1) Preprocessing

In the long read mode, the reads, quality values, and read identifiers are separated and compressed in blocks (reads and quality values using BSC, identifiers using specialized identifier compressor described above). By default, the block length for long reads is set to 10,000 reads. The read lengths are also stored as 32-bit integers in a separate stream which is compressed using BSC. Preprocessing is directly followed by the Tar stage for long reads. In the order-preserving mode, the quality values and read IDs are compressed in blocks (quality values using BSC, identifiers using specialized identifier compressors). QVZ quantization is applied before quality compression if the corresponding flag is specified. By default, the block length for short reads is set to 256,000 reads. The reads are written to temporary files after separating out the reads containing the character “N”. The reads containing “N” are considered directly in the “Encode reads” stage. In the order non-preserving mode, the quality values and read IDs are written to temporary files. The reads are handled exactly as in the order-preserving mode. If the Illumina binning or binary thresholding flag is activated, the qualities are binned before compression/writing to a temporary file.

### (2) Reordering Reads

This step in read compression is based on HARC with several extensions and improvements. In this step, SPRING reorders the reads so that they are approximately ordered according to their position in the genome. The reordering is done in an iterative manner: given the current read, the sequence-data compression method disclosed herein tries to find a read which matches the prefix or the suffix of the current read with a small Hamming distance. To do this efficiently, a hash table is used which indexes the reads according to certain substrings of the read. The sequence-data compression method disclosed herein makes the following improvements to this stage:
HARC searched for matching reads in only one direction (matching the suffix of the current read). On the other hand, the sequence-data compression method disclosed herein looks for matches in both directions. This boosts read compression by 5% to 10% on most datasets
While HARC only supported fixed length reads of maximum length 255, the sequence-data compression method disclosed herein adds support to variable length short reads of maximum length 1011. For this, the sequence-data compression method disclosed herein stores an array containing the read lengths which is used to ensure that the Hamming distance between reads of different lengths is computed correctly.
As those skilled in the art will appreciate, most of the time in the reordering stage may be spent on a small fraction of remaining reads and the attempts to find matches to these reads usually fails. To save time in this step, the sequence-data compression method disclosed herein introduces early stopping to this stage. Each thread maintains the fraction of unmatched reads in the last one (1) million reads and stops looking for matches once this fraction crosses a certain threshold (for example, 50%). Since this stage is the most time-consuming step in the sequence-data compression method disclosed herein, early stopping can reduce compression times by as much as 20% without affecting the compression ratio

### (3) Encoding Reads

In this step, the sequence of reordered reads is used to obtain a majority-based reference sequence. The reference sequence is then used to encode the reordered reads. The final encoding includes the reference sequence, the positions of the reads in the reference sequence, and the mismatches of reads with respect to the reference sequence. An index mapping the reordered reads to their position in the original FASTQ file is also stored. The sequence-data compression method disclosed herein provides the support for variable-length reads of lengths up to 1055. This stage produces a majority-based reference sequence and encoded streams for reads aligned to the reference. A small fraction of reads usually remains unaligned to the reference to the reference and are stored separately. However, the encoded streams do not correspond to the original order of reads in the FASTQ file. Furthermore, the reordering and encoding stages of the sequence-data compression method disclosed herein consider the paired end FASTQ files as a single end FASTQ file obtained by concatenating the two files. Thus, for both the order preserving and order non-preserving modes, these streams may be transformed using the information in the index mapping of the reordered reads to their position in the original file. This is done in the next two steps.

### (4) Paired End Order Encoding

This step is used only in the order non-preserving mode. A new ordering of the reads is generated which preserves the pairing information while achieving the optimal compression. This step generates an index mapping of the reordered reads to their position in the new ordering. The reads in file 1 (see FIG. 5) are kept in the same order as obtained after the previous stage (encoding reads), that is, the reads in file 1 are sorted according to their position in the majority-based reference. This allows storing of the positions of these reads in the majority-based reference using delta-coding leading to improved compression. The ordering of the reads in file 2 (see FIG. 5) is automatically determined by the ordering of reads in file 1 (since pairing information is preserved). For single end files (in the order non-preserving mode), the reads are kept in the same order as obtained after the encoding stage (i.e., sorted according to their position in the majority-based reference).

### (5) Reorder and Compress Read Streams

In this step, the final encoded streams are generated and compressed in blocks using BSC. For this purpose, first, the streams generated by the encoding stage are loaded into the memory, which are then reordered according to the mode. In the order-preserving mode, the streams are ordered according to the original order of reads in the FASTQ files. In the order non-preserving mode, the streams are ordered according to the new order generated in the paired end order encoding step. The final streams are described below:
seq: seq stores the majority-based reference sequence. This is packed into a two (2) bits/base representation before compression.
flag: indicates whether the reads are aligned or not as well as the distance between them on the reference.
   flag set to zero (0): Both reads aligned and gap between alignment positions is less than 32,767 (for single end datasets, flag 0 means that the read is aligned).
   flag set to one (1): Both reads aligned and gap between alignment positions is greater than or equal to 32,767.
   flag set to two (2): Both reads unaligned (for single end datasets, flag 2 means that the read is unaligned).
   flag set to three (3): read 1 of pair aligned, read 2 unaligned.
   flag set to four (4): read 1 of pair unaligned, read 2 aligned.
pos: in the order-preserving mode, pos stores the position of the first read of the pair (and possibly the second read) on the reference using eight (8) bytes. If flag is zero (0) or three (3), only the position of the first read is stored. If flag is one (1), positions of both the first and the second reads are stored. If flag is two (2), nothing is stored. In the order non-preserving mode, the position of the first read of the pair is stored as the difference from the first read of the previous pair (except for the first pair in the block). Note that the difference is always positive because of the way the new order is defined in the paired end order encoding step. This difference is stored as a two (2) byte unsigned integer as long as it is less than 65,535; otherwise, 65,535 is stored using two (2) bytes followed by the actual difference using eight (8) bytes. Storing differences rather than the absolute position allows SPRING to achieve significantly better compression in the order non-preserving mode.

pos pair: for paired end datasets, pos pair store the gap between the paired reads on the reference using a 16-bit signed integer when the flag is zero (0). Since the paired reads are sequenced from nearby portions of the genome (paired reads are typically separated by 50 to 250 bases), they are likely to appear close in the reference. Using a separate stream for the gap between the paired reads allows us to exploit this fact.
noise: noise stores the noisy bases in the aligned reads with respect to the reference. The encoding depends on both the base in the reference and in the read, allowing exploiting of the fact that certain errors are more likely in Illumina sequencing. For example, the most probable transitions for each reference symbol are encoded as zero (0), next most probable transitions as one (1), and so on. This leads to more 0's in the encoded stream leading to better compression. A newline character separates the noise for consecutive reads.
noisepos: noisepos stores the position of the noisy bases encoded in the noise stream, which are delta encoded to exploit the fact that most sequencing errors occur towards the end of the read. The delta coded noise positions are stored as 16-bit unsigned integers.
RC: RC stores the orientation (forward/reverse) of aligned reads with respect to the reference. If flag is zero (0), RC does not store the orientation of the second read in the pair (see RC pair stream).
RC pair: for paired end datasets, RC pair store the relative orientation of the second read with respect to the first read when the flag is zero (0). If the paired reads have opposite orientation, store zero (0); otherwise store one (1). Since the paired end reads have opposite orientation of the genome, we expect to get mostly 0's in this stream and hence this stream is highly compressible.
unaligned: unaligned stores the unaligned reads without any encoding.
length: length stores the read lengths as 16-bit unsigned integers.

Although embodiments have been described above with reference to the accompanying drawings, those of skill in the art will appreciate that variations and modifications may be made without departing from the scope thereof as defined by the appended claims.

1. A method of compressing genomic data, the method comprising:
aligning the genomic data with reference data;
obtaining difference between the genomic data and the reference data; and
compressing the difference using a statistical compression method to obtain compressed genomic data.

2. The method of claim 1, wherein the statistical compression method is an arithmetic coding method.

3. The method of claim 1 further comprising:
processing the difference using one or more statistical modeling methods; and
wherein said compressing the difference using the statistical compression method comprises:
compressing the processed difference using the statistical compression method.

4. The method of claim 1 further comprising:
assembling a plurality of reads to form the reference data.

5. The method of claim 1 further comprising:
storing compressed genomic data in a blockchain.

6. One or more non-transitory computer-readable storage media comprising computer-executable instructions for compressing genomic data, wherein the instructions, when executed, cause a processing structure to perform the method of claim 1.

7. (canceled)

8. The one or more non-transitory computer-readable storage media of claim 6, wherein the statistical compression method is an arithmetic coding method.

9. The one or more non-transitory computer-readable storage media of claim 6, wherein the instructions, when executed, cause a processing structure to perform further actions comprising:
processing the difference using one or more statistical modeling methods; and
wherein said compressing the difference using the statistical compression method comprises:
compressing the processed difference using the statistical compression method.

10. The one or more non-transitory computer-readable storage media of claim 6, wherein the instructions, when executed, cause a processing structure to perform further actions comprising:
assembling a plurality of reads to form the reference data.

11. The one or more non-transitory computer-readable storage media of claim 6, wherein the instructions, when executed, cause a processing structure to perform further actions comprising:
storing compressed genomic data in a blockchain.

12. One or more processors functionally coupled to one or more non-transitory computer-readable storage media, wherein the one or more non-transitory computer-readable storage media comprise computer-executable instructions for compressing genomic data, wherein the instructions, when executed, cause a processing structure to perform the method of configured for performing the method of claim 1.

13. The one or more processors of claim 12, wherein the statistical compression method is an arithmetic coding method.

14. The one or more processors of claim 12, wherein the instructions, when executed, cause a processing structure to perform further actions comprising:
processing the difference using one or more statistical modeling methods; and
wherein said compressing the difference using the statistical compression method comprises:
compressing the processed difference using the statistical compression method.

15. The one or more processors of claim 12, wherein the instructions, when executed, cause a processing structure to perform further actions comprising:
assembling a plurality of reads to form the reference data.

16. The one or more processors of claim 12, wherein the instructions, when executed, cause a processing structure to perform further actions comprising:
storing compressed genomic data in a blockchain.
